(19) United States
(12) Patent Application Publication
(10) Pub. No.: US 2023/0323440 A1
(43) Pub. Date: Oct. 12, 2023

(54) METHOD AND SYSTEM FOR SIZING A POPULATION OF NUCLEIC ACID FRAGMENTS USING A DIGITAL ASSAY

(71) Applicant: Bio-Rad Laboratories, Inc., Hercules, CA (US)

(72) Inventors: Stephen C. BOLARIS, Livermore, CA (US); Meiye WU, Tracy, CA (US); Mark WHITE, Orinda, CA (US); Dawne N. SHELTON, San Ramon, CA (US)

(21) Appl. No.: 18/189,723

(22) Filed: Mar. 24, 2023

Related U.S. Application Data
(60) Provisional application No. 63/323,314, filed on Mar. 24, 2022.

(51) Int. Cl. G16B 25/00; C12Q 1/6851; G16B 40/20

(52) U.S. Cl. CPC C12Q 1/6851; G16B 25/00; G16B 40/20

(57) ABSTRACT

Methods, systems, and computer-readable media for sizing a population of nucleic acid fragments. In an exemplary method of sizing a population of nucleic acid fragments provided by a sample, partitions may be formed, each containing a portion of the sample. Each target of at least two targets may be present in only a fraction of the nucleic acid fragments. Amplification reactions may be performed for each of the at least two targets in the partitions. Amplification data may be collected for the at least two targets from the partitions. Target-defined subsets of the partitions may be enumerated using the amplification data. At least one length characteristic of the population of nucleic acid fragments may be predicted using results of enumerating.

### CROSS-REFERENCE TO PRIORITY APPLICATION

This application is based upon and claims the benefit under 35 U.S.C. § 119(e) of U.S. Provisional Patent Application Serial No. 63/323,314, filed Mar. 24, 2022, which is incorporated herein by reference in its entirety for all purposes.

### INTRODUCTION

A biological product, such as a monoclonal antibody, a therapeutic protein, a hormone, or the like, can be biosynthesized on a commercial scale by cells cultured in a bioreactor. After biosynthesis, the biological product is isolated from the cells and then processed and purified to remove potentially harmful byproducts. One of these byproducts is fragmented DNA, which may have the potential to be oncogenic, immunogenic, or infective in certain scenarios. To minimize the risk for use in humans, the Food and Drug Administration (FDA) and the World Health Organization (WHO) have established content limits (e.g., less than 10 ng/dose) and size limits (e.g., a median length of less than 200 base pairs) on the residual DNA present in a biological product. A size limit makes the residual DNA unlikely to contain an entire gene. Each batch of the biological product must be tested to ensure that the residual DNA therein does not exceed the size limit. However, available techniques for sizing nucleic acid fragments in a sample suffer from various disadvantages, such as poor sensitivity and specificity, low throughput, high cost, and/or difficulty of automation, among others.

### SUMMARY

The present disclosure provides methods, systems, and computer-readable media for sizing a population of nucleic acid fragments. In an exemplary method of sizing a population of nucleic acid fragments provided by a sample, partitions may be formed, each containing a portion of the sample. Each target of at least two targets may be present in only a fraction of the nucleic acid fragments. Amplification reactions may be performed for each of the at least two targets in the partitions. Amplification data may be collected for the at least two targets from the partitions. Target-defined subsets of the partitions may be enumerated using the amplification data. At least one length characteristic of the population of nucleic acid fragments may be predicted using results of enumerating.

### BRIEF DESCRIPTION OF THE DRAWINGS

FIG. 1 is a schematic flow diagram presenting selected aspects of an illustrative method and system for sizing a population of nucleic acid fragments using a digital assay for a first target and a second target of different length, performed with a sample-containing fluid in which the nucleic acid fragments are much larger than each target.

FIG. 2 is another schematic flow diagram presenting selected aspects of the method and system of FIG. 1, performed with a different sample-containing fluid that is more fragmented, such that the nucleic acid fragments are generally comparable in size to the first and second targets.

FIG. 3 is a graph illustrating an exemplary exponential relationship between the degree of sample fragmentation and the ratio of target levels for the first and second targets of FIGS. 1 and 2.

FIG. 4 is a schematic view of an exemplary configuration of targets and reagents for the method and system of FIG. 1, taken in the presence of a nucleic acid fragment containing both the first target and the second target linked to one another and illustrating amplification of both targets.

FIG. 5 is another schematic view of the configuration of FIG. 4, except taken in the presence of a nucleic acid fragment containing only the second target.

FIG. 6 is yet another schematic view of the configuration of FIG. 4, except taken in the presence a nucleic acid fragment containing only the first target.

FIG. 7 is a schematic flow diagram presenting selected aspects of an illustrative method and system for sizing a population of nucleic acid fragments using a digital assay similar to that of FIG. 1, except with the linked target configuration of FIG. 4, and performed with a sample-containing fluid in which the nucleic acid fragments are much larger than each target.

FIG. 8 is another schematic flow diagram using the digital assay and linked targets of FIGS. 4 and 7, except performed with a sample-containing fluid exhibiting a greater amount of fragmentation.

FIGS. 9-16 are scatter plots of amplification data collected from digital amplification assays performed in droplets (as the partitions), using the target and reagent configuration of FIGS. 4-6 on a series of standards each having a separately-measured, different median length of nucleic acid fragments.

FIG. 17 is a bar graph plotting target concentrations determined from the amplification data presented in the scatter plots of FIGS. 9-16, as a function of the median fragment length of each standard.

FIG. 18 is a plot of size predictions for benzonase-fragmented samples, where the size predictions were generated with a machine learning model based on logistic regression.

FIG. 19 is a flowchart listing exemplary steps of an illustrative method of sizing a population of nucleic acid fragments using a digital assay.

FIG. 20 is a flowchart listing exemplary steps of an illustrative method of generating a size prediction algorithm for sizing a population of nucleic acid fragments using a digital assay.

### DETAILED DESCRIPTION

Various aspects and examples of methods of sizing a population of nucleic acid fragments, as well as related systems and computer-readable media, are described below and illustrated in the associated drawings. Unless otherwise specified, a method, system, or computer-readable medium for sizing nucleic acid fragments, contains at least one of the structures, components, functionalities, and/or variations described, illustrated, and/or incorporated herein. Furthermore, unless specifically excluded, the process steps, structures, components, functionalities, and/or variations described, illustrated, and/or incorporated herein may be included in other similar methods, systems, and/or computer-readable media, including being interchangeable between disclosed examples. The following description of various examples is merely illustrative in nature and is in no way intended to limit the examples, their applications, or their uses. Additionally, the advantages provided by the examples described below are illustrative in nature and not all examples provide the same advantages or the same degree of advantage.

The present disclosure provides methods, systems, and computer-readable media for sizing a population of nucleic acid fragments using a digital assay. Further aspects of the present disclosure are described in the following sections: (I) definitions, (II) overview, (III) examples, components, and alternatives, (IV) illustrative combinations and additional examples, (V) advantages and benefits, and (VI) conclusion.

Features, functions, and advantages may be achieved independently in various examples of the present disclosure, or may be combined in yet other examples, further details of which can be seen with reference to the following description and drawings.

### I. Definitions

Technical terms used in this disclosure have meanings that are commonly recognized by those skilled in the art. However, the following terms may be further defined as follows.

An “amplicon” is a product of an amplification reaction. Copies of an amplicon may be generated by amplification of a target sequence, such that the amplicon corresponds to the target sequence (i.e., matches and/or is complementary to the target sequence). However, the sequence of the amplicon, such as at primer binding sites, may not exactly match and/or may not be perfectly complementary to the target sequence.

“Amplification” is a process whereby multiple copies are made of an amplicon matching, complementary, and/or otherwise corresponding to a target sequence. The process interchangeably may be called an amplification reaction. Amplification may generate an exponential increase in the number of copies as amplification proceeds. Typical amplifications may produce a greater than 100-fold or 1,000-fold increase in the number of copies of an amplicon. Exemplary amplification reactions for the methods disclosed herein may include a polymerase chain reaction (PCR) or a ligase chain reaction (LCR), each of which is driven by thermal cycling. The methods also or alternatively may use other amplification reactions, which may be performed isothermally, such as branched-probe DNA assays, cascade-RCA, helicase-dependent amplification, loop-mediated isothermal amplification (LAMP), nucleic acid based amplification (NASBA), nicking enzyme amplification reaction (NEAR), PAN-AC, Q-beta replicase amplification, rolling circle replication (RCA), self-sustaining sequence replication, strand-displacement amplification, and/or the like. Amplification may utilize a linear or circular template.

“Amplification reagents” are any reagents that promote generation of an amplicon by amplification of a target sequence. The reagents may include any combination of at least one primer pair for amplification of at least one target sequence, at least one label for detecting amplification of the at least one target sequence (e.g., at least one probe including a label and/or an intercalating dye as a label), at least one polymerase enzyme and/or ligase enzyme (which may be heat-stable), and nucleoside triphosphates (dNTPs and/or NTPs), among others.

A “carrier fluid” is a fluid that contacts partitions, optionally enclosing each partition. The fluid may be liquid or gas. The carrier fluid may be described as a continuous phase and the partitions therein as a dispersed phase. The carrier fluid may be immiscible with, and encapsulate each partition. In some examples, the carrier fluid may be an oil, such as including a fluorocarbon oil or a silicone oil.

“Complementary” means related by the rules of base pairing. A first nucleic acid polymer, or region thereof, is “complementary” to a second nucleic acid polymer if the first nucleic acid polymer or region is capable of hybridizing with the second nucleic acid polymer in an antiparallel fashion by forming a consecutive (uninterrupted) or nearly consecutive series of base pairs (e.g., at least 5, 6, 7, 8, 9, or 10 consecutive base pairs). The first nucleic acid polymer (or region thereof) is termed “perfectly complementary” to the second nucleic acid polymer if hybridization of the first nucleic acid (or region thereof) to the second nucleic acid polymer forms a consecutive series of base pairs using every nucleotide of the first nucleic acid polymer or region thereof. A “complement” of a first nucleic acid polymer or region thereof is a second nucleic acid polymer or region thereof that is perfectly complementary to the first nucleic acid polymer or region thereof. The “complementarity” between a first nucleic acid polymer (or region thereof) and a second nucleic acid polymer (or region thereof) refers to the number or percentage of base pairs that can be formed when the first nucleic acid polymer (or region thereof) is optimally aligned for hybridization in an antiparallel fashion with the second nucleic acid polymer (or region thereof). A first nucleic acid polymer or region thereof that is complementary to a second nucleic acid polymer or region thereof generally has a complementarity of at least 80% or 90%.

“Comprising,” “including,” and “having” (and conjugations thereof) are used interchangeably to mean including but not necessarily limited to, and are open-ended terms not intended to exclude additional, unrecited elements or method steps.

A “computer-readable medium” includes any removable/non-removable, volatile/nonvolatile storage medium that is readable by a machine. Exemplary types of computer-readable media include magnetic media, optical media, and electronic media, among others, such as hard drives, optical discs, magnetic tapes, magnetic discs, solid state random access memory (RAM), solid state read only memory (ROM), and the like. The computer-readable medium may be transitory or non-transitory.

“Coupled” means to be in such relation that the performance of one influences the performance of the other, may include being connected, either permanently or releasably, whether directly or indirectly through intervening components, and is not necessarily limited to physical connection(s).

A “digital assay” is an investigative procedure(s) capable of detecting single copies of an analyte in a set of partitions. A “digital amplification assay” is a digital assay that utilizes an amplification reaction(s) to facilitate detection of single copies of a target(s). A digital assay may be performed with any suitable number of partitions that gives a statistically significant result, such as at least twenty, one-hundred, one-thousand, or ten-thousand, among others.

A “droplet” is a small volume of liquid encapsulated by an immiscible fluid (e.g., encapsulated by an immiscible liquid, which may form a continuous phase of an emulsion). The immiscible liquid may include oil and/or may be composed predominantly of oil. Droplets disclosed herein may, for example, have an average volume of less than about 500 nL, 100 nL, 10 nL, or 1 nL, among others.

An “enumeration value” is any value that results from enumerating a target-defined subset of a set of partitions in a digital assay. The enumeration value may, for example, represent a number of partitions in the set of partitions that are positive for the presence of a given target or two or more given targets, negative for the presence of a given target or two or more given targets, positive for only a specified subset of one or more targets of a set of targets, negative for only a specified subset of one or more targets of a set of targets, or the like.

“Exemplary” means “illustrative” or “serving as an example.” Similarly, the term “exemplify” (or “exemplified”) means “to illustrate by giving an example.” Neither term implies desirability or superiority.

“First,” “second,” and similar terms are used to distinguish or identify various members of a group, or the like, in the order they are introduced in a particular context and are not intended to show serial or numerical limitation.

“Fluorescence” is optical radiation emitted in response to absorption of light. As used herein, fluorescence is intended to cover any form of photoluminescence, in which absorption of one or more photons promotes an electron to an excited state and leads to subsequent emission of a new photon, whether from a singlet state, a triplet state, or other state. The excited state produced by absorption may have any suitable lifetime.

A “fluorophore” is any atom, functional group, moiety, or substance capable of fluorescence.

“Fragmentation” or “fragmenting” of nucleic acid polymers (DNA and/or RNA) is the process of breaking the nucleic acid polymers into smaller pieces, termed “fragments,” which also are nucleic acid polymers (or monomers). The state of the nucleic acid polymers resulting from this process is called “fragmented” or “degraded.” Fragmentation/fragmenting may be performed by a physical process, a chemical process, or a combination thereof. Physical fragmentation generally is conducted by application of forces to the nucleic acid polymers sufficient to cause breakage. The forces may be hydrodynamic shear forces, which may be generated by any suitable approach, such as by sonication, pulses of acoustic energy, centrifugation, passage through a needle, point-sink shearing, or the like. Chemical fragmentation is conducted through a chemical reaction(s) using the nucleic acid polymers as a reactant. The chemical reaction(s) may be enzyme catalyzed, such as with an endonuclease. Exemplary chemical fragmentation is performed by a hydrolysis reaction that cleaves the phosphodiester backbone of nucleic acid polymers.

An “inverse” of a subset of a set of partitions is the remainder of the set. For example, if a first subset of the set of partitions is positive for a first target and negative for a second target, the inverse of the first subset is composed of all other partitions of the set, namely, partitions that are negative for the first target and the second target, partitions that are positive for the first target and the second target, and partitions that are negative for the first target and positive for the second target. Similarly, if a second subset of the set of partitions is positive for the second target and negative for the first target, the inverse of the second subset is composed of all other partitions of the set, namely, partitions that are negative for the first target and the second target, partitions that are positive for the first target and the second target, and partitions that are negative for the second target and positive for the first target. Furthermore, if each partition of a third subset of the set of partitions is positive for both the first target and the second target, the inverse of the third subset is composed of all other partitions of the set, namely, partitions that are negative for the first target and the second target, partitions that are positive for the first target and negative for the second target, and partitions that are negative for the first target and positive for the second target. The partition count for an inverse of a given subset of a set of partitions is equal to the total partition count for the set of partitions minus the partition count for the given subset.

A “label” is an identifying and/or distinguishing marker or identifier associated with a structure, such as a primer, probe, amplicon, partition, or the like. The label may be associated covalently with the structure, such as a label that is conjugated to an oligonucleotide, or associated non-covalently (e.g., by intercalation, hydrogen bonding, electrostatic interaction, encapsulation, etc.). Exemplary labels include optical labels, radioactive labels, magnetic labels, electrical labels, epitopes, enzymes, antibodies, etc. Optical labels are detectable optically via their interaction with light. Exemplary optical labels that may be suitable include fluorophores and quenchers, among others.

A “length characteristic” is any measure or description of the length of a population of nucleic acid fragments. For example, the length characteristic may be expressed with respect to base pairs/nucleotides, molecular weight, nanometers, or the like, and may be a mean length, a median length, a quartile length, or a quintile length, among others, of nucleic acid fragments in the population. In some cases, the length characteristic may be a classification with respect to one or more threshold sizes, such as a binary classification with respect to a threshold size, such as 200 nucleotides (e.g., less than (below), or greater than (above), or greater than or equal to, the threshold size). In some cases, the length characteristic may be a percentage of fragments (by number or mass) that are above or below a threshold size. The term “size” may be used herein as a synonym for length.

The methods of the present disclosure may predict any suitable combination of length characteristics for a population of nucleic acid fragments. The combination of length characteristics may include a median size estimate (e.g., in bp or nucleotides). The combination also may include an estimated confidence interval of the median (such as a 90%, 95%, or 99% confidence interval for the median), which can be calculated using Poisson statistics to provide upper and lower input values for a predictor function also used to estimate the median size. The combination also or alternatively may include a value for a percentage of the population of nucleic acid fragments that is less than or greater than (or equal to) a threshold size, such as 200 bp. The combination further may include a confidence interval (such as a 90%, 95%, or 99% confidence interval) for the percentage that is less than or greater than the threshold size.

A “predictor function” is any function that provides an output value for a dependent variable, based on one or more input values for one or more independent variables, where the output value represents a prediction from the one or more input values. A predictor function may include an expression having any suitable number of variable terms, a coefficient for each variable term, and a constant term. The expression may include 1, 2, 3, or more independent variables, an equal or greater number of variable terms including the independent variables, and a constant term. Values for coefficients and the constant term of the predictor function may be estimated by machine learning using a computer and a dataset composed of data points to which the predictor function with be fitted by estimating values for the coefficients and the constant term of the predictor function.

A “linear predictor function” has the following general form:

$$y = {β}_{1} {x}_{1} + ⋯ + {β}_{p} {x}_{p} + b \qquad \text{(1)}$$

Where the expression to the right of the equals sign has p variable terms and one constant term, where xₖ, for k= 1, ..., p, is the k-th independent (input/explanatory) variable, β₁, ..., βₚ are the respective coefficients of the independent variables (and variable terms), b is a constant term (an intercept), and y is the dependent (output) variable. The values of the independent variables for a given data point i are _{Xi1}, ..., xᵢₚ, and the value of the dependent variable for data point i is yᵢ. The linear predictor functions of the present disclosure may have only one (p=1), only two (p=2), only three (p=3), or more (p>3) independent variables. A linear predictor function with two or more independent variables is a linear combination of the independent variables. An input value for each independent variable is multiplied by the corresponding coefficient and the products are summed with one another (and with the intercept) to yield a value for the dependent variable. Values for the respective coefficients of the independent variables can be estimated from a dataset including data points, where each data point has a value for the dependent variable and an associated value for each independent variable. The process of estimating coefficients and/or an intercept can be performed by machine learning.

“Linear regression” is a linear approach for modeling the relationship between a dependent variable (an output variable (e.g., a scalar response)) and one or more independent variables (input variables (also called explanatory variables). Simple linear regression is performed with a linear predictor function having one independent variable and multiple linear regression with a linear predictor function having two or more independent variables. Linear regression creates a linear regression model by fitting a linear predictor function to a dataset composed of data points, where each data point has a value for the dependent variable and an associated value for each independent variable. The fitting process may involve estimating a value for each coefficient and a value of the intercept of the linear predictor function, such as by the method of least squares.

“Logistic regression” is an approach for modeling the relationship between a dependent variable (an output variable) having only a finite number of permitted values and one or more independent variables (input variables). Binary regression is logistic regression in which the dependent variable is a binary variable having only two permitted values (e.g., 0 or 1). Logistic regression may create a logistic regression model by fitting a linear predictor function of general form to a dataset composed of data points, where each data point has a value for the dependent variable and an associated value for each independent variable. The linear predictor function may be a linear combination of weighted input variables. The fitting process may involve estimating a value for each coefficient and a value for the intercept of the linear predictor function, such as by maximum likelihood estimation (MLE).

“Machine learning” is any computer-implemented procedure or set of procedures that generates a prediction algorithm based on collected data. Machine learning may involve estimating a value of each coefficient and a value of a constant (e.g., an intercept) of a predictor function.

A “nucleic acid polymer” is a molecule or molecular duplex of any length composed of naturally-occurring nucleotides (e.g., where the polymer is DNA and/or RNA), or a compound produced synthetically that can hybridize with DNA or RNA in a sequence-specific manner analogous to that of two naturally occurring nucleic acids, e.g., can participate in Watson-Crick base pairing interactions. A nucleic acid polymer may be composed of any suitable number of nucleotides, such as at least about 5, 10, 100, or 1000, among others.

A nucleic acid polymer may have a natural or artificial structure, or a combination thereof. Nucleic acid polymers with a natural structure, namely, deoxyribonucleic acid (DNA) and ribonucleic acid (RNA), generally have a backbone of alternating pentose sugar groups and phosphate groups. Each pentose group is linked to a nucleobase (e.g., a purine (such as adenine (A) or guanine (G)) or a pyrimidine (such as cytosine (C), thymine (T), or uracil (U))). Nucleic acid polymers with an artificial structure are analogs of natural nucleic acids and may, for example, be created by changes to the pentose and/or phosphate groups of the natural backbone and/or to one or more nucleobases. Exemplary artificial nucleic acid polymers include glycol nucleic acids (GNAs), peptide nucleic acids (PNAs), locked nucleic acids (LNAs), threose nucleic acids (TNAs), xeno nucleic acids (XNA), and the like.

The sequence of a nucleic acid polymer is defined by the order in which nucleobases are arranged along the backbone. This sequence generally determines the ability of the nucleic acid polymer to hybridize with another nucleic acid by hydrogen bonding. In particular, adenine pairs with thymine (or uracil) and guanine pairs with cytosine.

An “oligonucleotide” is a relatively short and/or chemically synthesized nucleic acid polymer. The length of an oligonucleotide may, for example, be 3 to 1000 nucleotides, among others. In some cases, an oligonucleotide may be labeled with at least one label, which may be conjugated to any suitable structure of the oligonucleotide. The at least one label may include at least one fluorophore and thus may be a fluorescent label. Each label may be conjugated to the oligonucleotide at any suitable position, including a 5′-end, a 3′-end, or intermediate the 5′- and 3′-ends.

“Optical radiation” means electromagnetic radiation in the optical spectrum, namely, ultraviolet light, visible light, and/or infrared light. The term “light” without modification has the same meaning as optical radiation.

“Partial occupancy” means present or contained in only a subset of a set of partitions. A set of partitions containing a target at partial occupancy means that at least one copy of the target is present in each partition of only a subset of the partitions. In other words, one or more of the partitions do not contain any copies of the target. Copies of the target may be distributed randomly among the partitions but may be in limited supply such that every partition fails to receive a copy of the target.

“Partitions” are discrete volumes of fluid (i.e., fluid volumes) that are isolated from one another (also called isolated volumes). Each partition of a set of partitions may contain a portion of the same sample. The partitions may be separated from one another by fluid (e.g., oil or air), a wall(s) of a device(s), or a combination thereof, among others. Accordingly, the partitions may be droplets of an emulsion, or volumes held by wells, chambers (e.g., nanochambers having a capacity of less than 1 µL), or tubes (e.g., microtubes having a diameter of less than 1 mm), among others. The partitions may be the same diameter as one another and/or may be composed of the same amount of fluid as one another.

A “partition count” is a value for the number of partitions in a specified group, such as a subset of partitions having a specified target content. A partition count may be an enumeration value.

The term “positive” when used to indicate a target content of a fragment, partition, or subset of a set of partitions indicates that the fragment, partition, or each partition of the subset contains (or at least appears and/or is deemed to contain) at least one copy of a given target or of each target of a given set of targets. The term “negative” when used to indicate a target content of a fragment, partition, or subset of partitions indicates that the fragment, partition, or each partition of the subset does not contain (or at least appears and/or is deemed not to contain) at least one copy of a given target or of each target of a given set of targets.

A “prediction algorithm” is a set of instructions followed by a machine to predict a characteristic or outcome. A “size prediction algorithm” is a prediction algorithm that predicts at least one length characteristic. The size prediction algorithm may be configured to utilize a predictor function, such as a linear predictor function, to calculate an output value representing a length characteristic of a population of nucleic acid fragments. In some cases, the size prediction algorithm may be configured to receive an input value for each independent variable of a predictor function, for which values of the coefficient(s) and intercept have been estimated, and calculate an output value for the dependent variable of the predictor function. In some cases, the size prediction algorithm may be configured to utilize at least two predictor functions to generate at least two respective output values for at least two independent variables representing different length characteristics, using the same input values.

A “primer” is an oligonucleotide (DNA/RNA or an analog thereof) capable of serving as a point of initiation of template-directed nucleic acid synthesis or ligation under appropriate reaction conditions (e.g., in the presence of a template to which the primer anneals, nucleoside triphosphates, and an agent for polymerization (such as a DNA or RNA polymerase or a reverse transcriptase), in an appropriate buffer and at a suitable temperature). The primer may have any suitable length, such as 5 to 500 nucleotides, among others. The primer may be a member of a “primer pair” including a “forward primer” and a “reverse primer” that define the ends of an amplicon generated in an amplification reaction. (The adjectives “forward” and “reverse” are arbitrary designations relative to one another.) The forward primer hybridizes with a complement of the 5′-end region of a template sequence to be amplified, and the reverse primer hybridizes with the 3′-end region of the template sequence. The term “primer binding site” refers to a portion of a template (or its complement) to which a primer anneals. The full sequence of the primer need not be perfectly complementary to the primer binding site, just sufficiently complementary to anneal under the conditions of the reaction. Accordingly, the primer may have a 3′-end region that is complementary to the primer binding site, and a 5′-end region that is not complementary to the primer binding site (and forms a “5′-tail”).

A “probe” is a labeled oligonucleotide configured to report the occurrence of an amplification reaction and/or formation of an amplicon by the amplification reaction. A probe may be a fluorescent probe including an oligonucleotide labeled with a fluorophore. A probe may be configured to hybridize with at least a portion of an amplicon generated by amplification. The probe (e.g., a hydrolysis probe) may be configured to hybridize with at least a portion of an amplicon during an annealing/extension phase of amplification cycles of an amplification reaction, or the probe (e.g., a molecular beacon probe or strand displacement probe) may be configured to hybridize with the amplicon after the amplification reaction has been completed, among others.

A “reagent” is any substance or agent used for any purpose in a reaction, except for the analyte (e.g., a population of nucleic acid fragments and/or a target(s)) being analyzed.

A “standard” is a sample containing nucleic acid fragments subjected (or to be subjected) to two different sizing techniques. One of the sizing techniques includes a digital assay for at least a pair of targets provided by the nucleic acid fragments of the standard.

“Substantially” means to be predominantly conforming to the particular dimension, range, shape, concept, or other aspect modified by the term, such that a feature or component need not conform exactly, so long as it is suitable for its intended purpose or function. For example, a “substantially cylindrical” object means that the object resembles a cylinder, but may have one or more deviations from a true cylinder.

A “target” (also called a “target sequence”) is a nucleic acid polymer sequence (DNA and/or RNA) of any suitable length that is amplified in an amplification reaction. Exemplary target sequences are about 20-1000 nucleotides, or about 30-500 nucleotides, among others.

A “target-defined subset” of a set of partitions is composed of partitions having a predefined target content (positivity/negativity) of one or more targets.

A “template” is a nucleic acid polymer including a target sequence and/or a complement thereof.

### II. Overview

This section provides an overview of the methods, systems, and computer-readable media for sizing a population of nucleic acids using a digital assay.

An exemplary method of sizing a population of nucleic acid fragments of a sample is provided. In the method, partitions may be formed, each containing a portion of the sample. Each target of at least two targets may be present in only a fraction of the nucleic acid fragments. Amplification reactions may be performed for each of the at least two targets in the partitions. Amplification data may be collected for the at least two targets from the partitions. Target-defined subsets of the partitions may be enumerated using the amplification data. At least one length characteristic of the population of nucleic acid fragments may be predicted using results of enumerating.

An exemplary method of obtaining a size prediction algorithm for sizing a population of nucleic acid fragments in a sample is provided. In the method, a series of standards may be provided. Each standard of the series may have a different degree of nucleic acid fragmentation and may include at least two targets. Sets of partitions may be formed. Each set of partitions may contain portions of one of the standards and may contain each of the at least two targets at partial occupancy. Amplification reactions for each of the least two targets may be performed in each set of partitions. Amplification data may be collected for the at least two targets from each set of partitions. Target-defined subsets of each set of partitions may be enumerated using the amplification data. For each standard, a length of nucleic acid fragments present in the standard may be measured. The size prediction algorithm may be generated by machine learning based on results of enumerating and measuring.

A computer-readable medium is provided. The computer-readable medium contains program instructions for sizing nucleic acid fragments in a sample. Execution of the program instructions by one or more processors of a computer system causes the one or more processors to perform a method. In the method, at least one length characteristic of a population of nucleic acid fragments may be predicted using values obtained by, or derived from, enumerating target-defined subsets of partitions in a digital amplification assay for at least two targets, The partitions may contain each of the at least two targets at partial occupancy. Each of the at least two targets may be provided by the population of nucleic acid fragments.

A system for sizing a population of nucleic acid fragments is provided. The system may comprise a first primer pair for amplifying a first target and a second primer pair for amplifying a second target. The system also may comprise a computer-readable medium containing program instructions executable by one or more processors of a computer system to predict at least one length characteristic of the population of nucleic acid fragments using values obtained or derived at least partially from enumerating target-defined subsets of partitions in a digital amplification assay. Each partition may contain the first primer pair and the second primer pair. The partitions may contain each of the first target and the second target at partial occupancy. The first target and the second target may be provided by the population of nucleic acid fragments.

### III. Examples, Components, and Alternatives

The following subsections, A-F, relate to methods, systems, and computer-readable media for sizing a population of nucleic acids using a digital assay. The examples in these subsections are intended for illustration and should not be interpreted as limiting the entire scope of the present disclosure. Each subsection may include one or more distinct examples, and/or contextual or related information, function, and/or structure.

### A. Digital Assay Method and System For Sizing a Population of Nucleic Acid Fragments

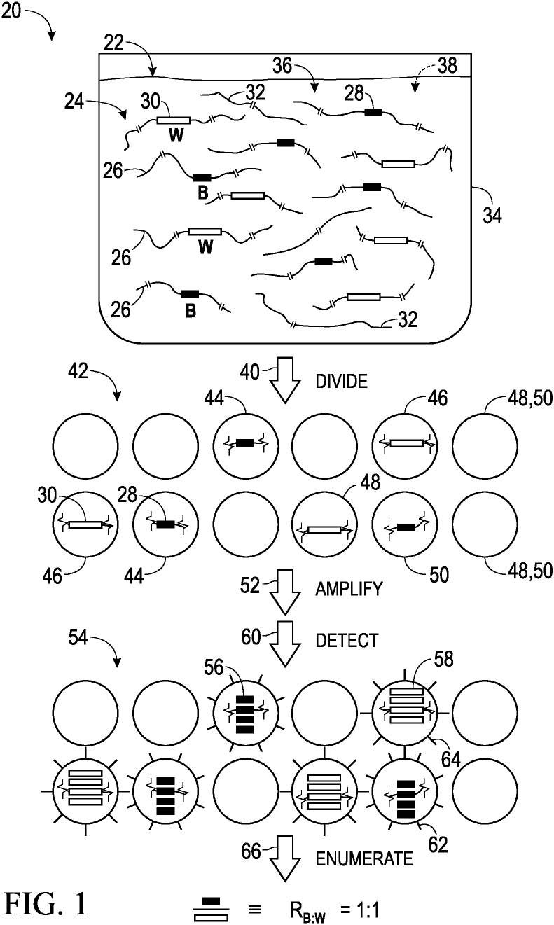

This subsection describes an illustrative method and system for sizing a population of nucleic acid fragments using a digital assay for a pair of targets of different length; see FIGS. 1-3.

FIG. 1 shows a schematic flow diagram 20 for performing an assay on a sample-containing fluid 22 to predict at least one length characteristic of a population 24 of nucleic acid fragments 26 therein. Nucleic acid fragments 26 provide copies of a first target 28 (interchangeably called target B; black boxes) and copies of a second target 30 (interchangeably called target W; white boxes). Only a fraction of nucleic acid fragments 26 contains first target 28, and only a fraction of the nucleic acid fragments contains second target 30. In most cases, a large majority of population 24 is composed of target-negative fragments 32, such that the fraction of population 24 containing one or both targets 28, 30 is only a small fraction of the total population (e.g., less than 1%, 0.1%, or 0.01%, among others). However, only a small number of target-negative fragments 32 are depicted in FIG. 1 for simplification.

Sample-containing fluid 22 may be prepared as a continuous phase held by a container 34. The continuous phase may include a sample 36 that provides population 24, and any suitable reagents for performing a digital assay. These reagents may include amplification reagents 38, such as amplification primers for each target 28, 30, a probe and/or label to enable detection of amplification, nucleotide monomers (e.g., dNTPs) for primer extension, an enzyme to catalyze amplification, and the like.

The sample-containing fluid 22 is divided, indicated by a dividing arrow 40, to form partitions 42. Each partition 42 is an isolated volume of sample-containing fluid 22, and includes a portion of sample 36 and each amplification reagent 38. Accordingly, each partition 42 may contain a plurality of target-negative fragments 32 from sample-containing fluid 22, although none are expressly shown here. The partitions collectively are a dispersed phase generated, at least in part, from a continuous phase. In some examples, each partition may be formed, at least in part, by fusing fluid volumes with one another. Only a small number of partitions is shown here for simplification.

Only a subset of partitions 42 contains at least one copy of one or both targets 28, 30. A first subset of the partitions are first-target partitions 44 containing at least one copy of first target 28. A second subset of the partitions are second-target partitions 46 containing at least one copy of second target 30. A third subset of the partitions are first-target-negative partitions 48 containing no copies of first target 28. A fourth subset of the partitions are second-target-negative partitions 50 containing no copies of second target 30. The first, second, third and fourth subsets may overlap with one another. In the example depicted in FIG. 1, the first target and the second target are not linked to one another and are distributed among the partitions independently of one another. With this configuration, colocalization of the two targets to the same partitions occurs by chance, in a concentration-dependent manner. For simplification, no partitions containing both targets 28, 30 are depicted, so the first and second subsets of partitions are nonoverlapping in this example, but in other examples these subsets of first-target partitions 44 and second-target partitions 46 do overlap one another. In some examples, the first target and the second target are tightly linked to one another and are colocalized efficiently to the same partitions, absent substantial fragmentation (e.g., see Subsection B). As another simplification, no partitions are shown to contain two or more copies of one of targets 28, 30. In other examples, multiple copies of each target are present in one or more of the partitions.

A respective amplification reaction for each target 28, 30 is performed in partitions 42, indicated by an amplifying arrow 52. As shown for post-amplification partitions 54, a first amplification reaction produces copies of a first amplicon 56 corresponding to first target 28, and a second amplification reaction produces copies of a second amplicon 58 corresponding to second target 30. First amplicon 56 is produced only in first-target partitions 44, and second amplicon 58 is produced only in second-target partitions 46.

The occurrence of each amplification reaction is detected for individual partitions 54, indicated by a detecting arrow 60, to collect amplification data. In the depicted example, a first fluorescence 62 and a second fluorescence 64 are detected from post-amplification partitions 54. The intensity of first fluorescence 62 detected from each partition 54 indicates whether the first amplification reaction has occurred in the partition, and the intensity of second fluorescence 64 indicates whether the second amplification reaction has occurred in the partition.

Partitions 54 are enumerated with respect to the presence/absence (positivity/negativity) of each target 28, 30, indicated by an enumerating arrow 66. Enumerating may, for example, include determining a partition count of partitions negative for the first target, a partition count of partitions positive for the first target, a partition count of partitions negative for the second target, a partition count of partitions positive for the second target, and/or a total number of the partitions.

In some cases, values obtained by enumerating may be used to derive other values. For example, enumeration values may be used to calculate a concentration value for each target (e.g., as copies per unit volume), a fraction of the partitions positive for each target (i.e., fraction-positive values for the targets), a fraction of the partitions negative for each target (i.e., fraction-negative values for the targets), normalized partition counts (e.g., scaled according to a predefined total partition count), a target ratio value(s) (e.g., a ratio of a first-target partition count to a second-target partition count, or vice versa), and/or the like.

Values obtained by, or derived at least in part from, enumerating may be utilized in various ways. In some cases, any of these values may be inputs for a size prediction algorithm that uses the inputs to predict at least one length characteristic of the sample (e.g., see Subsection D). In other cases, the sample may be a standard having a separately-measured (and, optionally, more directly measured) length characteristic, and any of these values may be used, along with corresponding values from other standards having a separately-measured length characteristic, to generate such a size prediction algorithm (e.g., see Subsection C).

As an illustrative example, the results of enumerating may be used to determine a target ratio (e.g., a concentration ratio, a partition count ratio, or the like) of the first target to the second target in sample-containing fluid 22. In FIG. 1, the nucleic acid polymers of sample 36 are not excessively fragmented, such that the median/mean length of nucleic acid fragments 26 is much greater than the lengths of first and second targets 28, 30. In this particular example, first target 28 and second target 30 have the same copy number in a genome or transcriptome represented by sample 36. For simplification, and because the sample is not excessively fragmented, only full-length, intact copies of first target 28 and second target 30 are shown to be present in sample-containing fluid 22, and are assumed to be amplified and detected with the same efficiency. As a result, copies of the first target (B) and second target (W) are determined in the assay to have a target ratio (R_{B}:W) of 1 (i.e., 1:1).

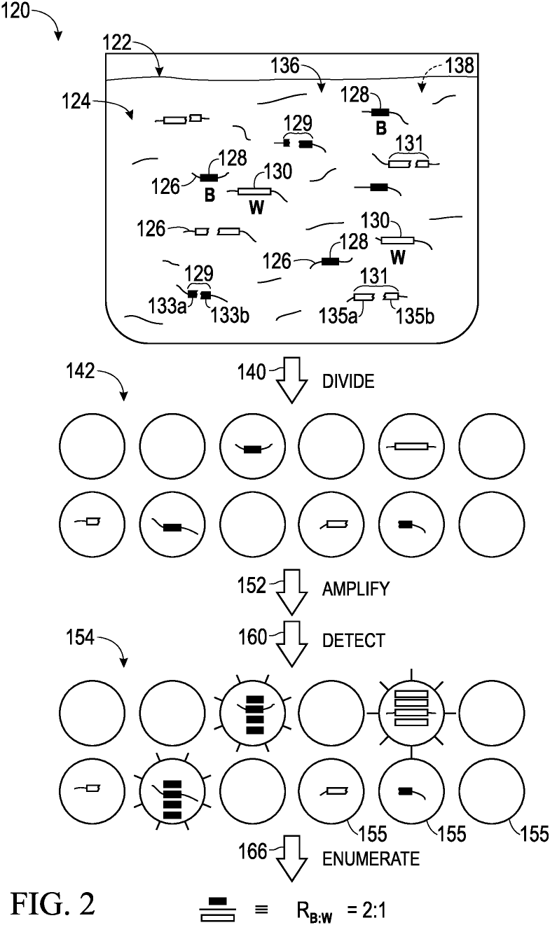

FIG. 2 shows another schematic flow diagram 120 for an assay very similar to the assay of FIG. 1. The assay of FIG. 2 is performed on a sample-containing fluid 122 representing the same genome or transcriptome as in FIG. 1, using amplification reagents 138 identical to amplification reagents 38 of FIG. 1. However, sample-containing fluid 122 has a population 124 of nucleic acid fragments 126, provided by a sample 136, that are substantially more fragmented than nucleic acid fragments 26 (compare sample-containing fluids 22 and 122 in FIGS. 1 and 2). In other words, the median/mean length of nucleic acid fragments 126 from sample 136 is much less than that of nucleic acid fragments 26 from sample 36, and is comparable to the length of first and second targets128, 130.

Nucleic acid fragments 126 provide unbroken and broken forms of each target. Intact first target 128 (unbroken target B; black boxes) is identical to first target 28. A broken first target 129 (broken target B; disrupted black boxes) is created by breaking intact first target 128. An intact second target 130 (unbroken target W; white boxes) is identical to second target 30. Finally, a broken second target 131 (broken target W; disrupted white boxes) is created by breaking intact second target 130.

The steps described above for flow diagram 20 of FIG. 1 are performed. Sample-containing fluid 122 is divided, indicated by a dividing arrow 140, to form partitions 142. A respective amplification reaction for each target is performed in partitions 142, indicated by an amplifying arrow 152. As shown for post-amplification partitions 154, intact first and second targets 128, 130 are amplifiable and detectable using amplification reagents 138. In contrast, broken first and second targets 129, 131 are not substantially amplifiable and/or detectable in the assay. For example, exponential amplification of a broken first target or broken second target does not occur in partitions 142 using a pair of primers for the target, because breakage separates the binding sites for the pair of primers to a pair of separate nucleic acid fragments 126. Stated differently, initial breakage divides a copy of an intact first target 128 or an intact second target 130 into a pair of first target fragments 133a, 133b or second target fragments 135a, 135b, each having a binding site for only one primer of the corresponding pair of primers. Partitions 142 that receive only a target fragment 133a, 133b, 135a, or 135b, or only any combination of two or more of these target fragments, behave as target-negative partitions 155 for amplification and detection.

Post-amplification partitions 154 are enumerated with respect to the presence/absence (positivity/negativity) of each intact target 128, 130, indicated by an enumerating arrow 166, as described above for flow diagram 20 of FIG. 1. The amount/concentration of each unbroken target is less than in FIG. 1 due to the increased fragmentation of nucleic acid polymers in sample 136. However, second target 130 is significantly longer than first target 128 (e.g., at least 25%, 50%, 75%, or 100% longer). For this reason, the frequency of second target breakage is greater than the frequency of first target breakage. In the present simplified example, the amount of fragmentation of sample 136 has increased the measured target ratio (R_{B}:W) of the first target (B) to the second target (W) to 2 (i.e., 2:1).

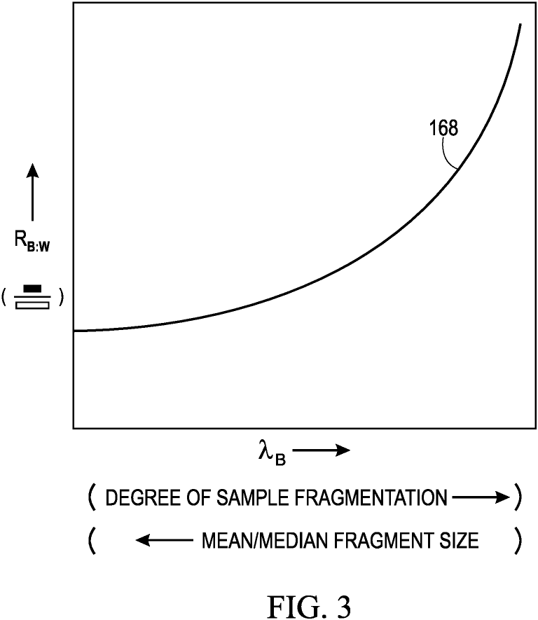

FIG. 3 shows an exemplary graph plotting a generalized exponential relationship between the degree of sample fragmentation and the ratio of unbroken target levels for a pair of targets, B (black) and W (white), of different length. The exponential relationship can be derived as shown below in Equations 2-22.

Equations 2-8 define various variables and relationships:

$$B ≡ B l a c k t a r g e t \qquad \text{(2)}$$

$$W ≡ W h i t e t a r g e t \qquad \text{(3)}$$

$${L}_{B} ≡ L e n g t h o f B \qquad \text{(4)}$$

$${L}_{W} ≡ L e n g t h o f W \qquad \text{(5)}$$

$${L}_{W} > {L}_{B} \qquad \text{(6)}$$

$$x ≡ R a t i o o f W : B t a r g e t l e n g t h s = \frac{{L}_{W}}{{L}_{B}} \qquad \text{(7)}$$

$${λ}_{t} ≡ M e a n n u m b e r o f b r e a k s i n a t a r g e t \left(t\right) \qquad \text{(8)}$$

The mean number of breaks in target W is assumed to scale with the mean number of breaks in target B, according to the ratio of target lengths, x, as shown in Equation 9:

$${λ}_{W} = x {λ}_{B} \qquad \text{(9)}$$

Equations 10-18 define further variables and relationships:

$${N}_{B 0} ≡ N u m b e r o f u n b r o k e n B w h e n {λ}_{B} = 0 \qquad \text{(10)}$$

$${N}_{W 0} ≡ N u m b e r o f u n b r o k e n W w h e n {λ}_{W} = 0 \qquad \text{(11)}$$

$${N}_{B i} ≡ N u m b e r o f u n b r o k e n B w h e n {λ}_{B} = i \qquad \text{(12)}$$

$${N}_{W i} ≡ N u m b e r o f u n b r o k e n W w h e n {λ}_{B} = i \qquad \text{(13)}$$

$$K ≡ x - 1 \qquad \text{(14)}$$

$${R}_{B : W} = \frac{{N}_{B i}}{{N}_{W i}} = R a t i o o f u n b r o k e n B t o u n b r o k e n W \qquad \text{(15)}$$

$${f}_{t} ≡ f r a c t i o n o f t a r g e t \left(t\right) t h a t i s b r o k e n \qquad \text{(16)}$$

$${N}_{B i} = {N}_{B 0} \left(1 - {f}_{B}\right) \qquad \text{(17)}$$

$${N}_{W i} = {N}_{W 0} \left(1 - {f}_{W}\right) \qquad \text{(18)}$$

Substituting for N_{B}i and Nwi in Equation 14 from Equations 17 and 18 gives Equation 19:

$${R}_{B : W} = \frac{{N}_{B 0} \left(1 - {f}_{B}\right)}{{N}_{W 0} \left(1 - {f}_{W}\right)} \qquad \text{(19)}$$

The breaks in each target are expected to have a Poisson distribution as shown in Equation 20:

$$P r o b a b i l i t y o f j b r e a k s i n a t a r g e t ≡ P \left(j\right) = \frac{{λ}^{j} {e}^{- λ}}{j !} \qquad \text{(20)}$$

Therefore, the fraction of each target that is broken (i.e., having least one break) is given by Equation 21:

$${f}_{t} = 1 - P \left(∅\right) = 1 - \frac{{λ}^{∅} {e}^{- λ}}{∅ !} = 1 - {e}^{- λ} \qquad \text{(21)}$$

Substituting for f_{B} and fw in Equation 18 using Equation 21, and simplifying, gives Equation 22:

$${R}_{B : W} = \left(\frac{{N}_{B 0}}{{N}_{W 0}}\right) {e}^{K {λ}_{B}} \qquad \text{(22)}$$

FIG. 3 shows a graph plotting target ratio R_{B}:w as a function of the mean number of breaks in target B, λ_{B}, based on Equation 22. In a resulting curve 168, R_{B}:_{W} increases exponentially for a sample with increasing fragmentation of nucleic acid polymers (and thus decreasing mean/median fragment length) in the sample, at least over a range of degrees of fragmentation. Accordingly, the target levels of a pair (or more) of targets change relative to one another as a function of the mean/median fragment length and provide size information about nucleic acid fragments in a sample, as explained further below.

### B. Illustrative target/reagent configuration

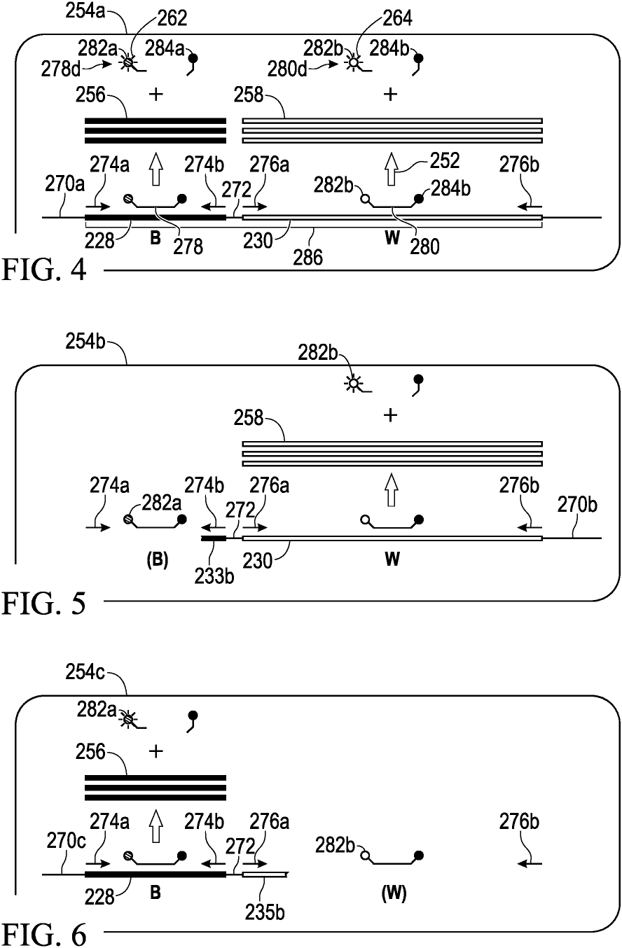

This subsection describes an exemplary configuration of linked targets and corresponding reagents for performing the digital assay of Subsection A, and exemplary flow diagrams for the digital assay performed with sample-containing fluids containing the linked targets and representing different degrees of nucleic acid fragmentation; see FIGS. 4-8.

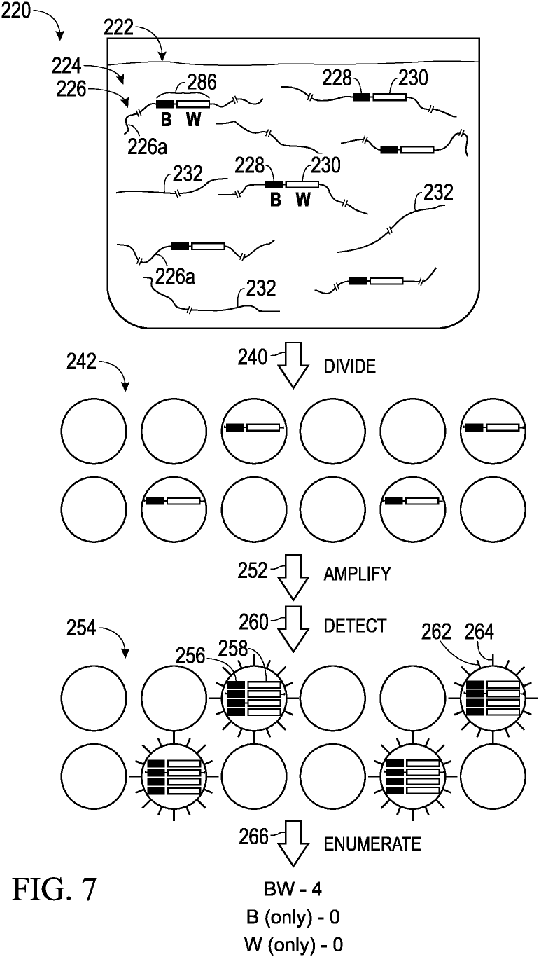

FIGS. 4-6 schematically illustrate three partitions 254a-254c containing respective nucleic acid fragments 270a-270c in the assay of FIGS. 1 and 2. Each nucleic acid fragment 270a-270c includes at least one unbroken target of a pair of linked targets. The linked targets are a first target 228 (unbroken target B) and a second target 230 (unbroken target W) of different length. Nucleic acid fragment 270a includes both unbroken targets B and W (see FIG. 4). Nucleic acid fragment 270b includes target W but only a broken part 233b of target B (see FIG. 5). Nucleic acid fragment 270c includes target B but only a broken part 235a of target W. Targets B and W may have any of the lengths described elsewhere herein, such as in Subsection A.

Partition 254a of FIG. 4 contains nucleic acid fragment 270a in which unbroken targets B and W are connected to one another by a spacer 272 (interchangeably called a spacer sequence). Spacer 272 may have any suitable length, such as at least 1, 2, 5, 10, 20, 50, or 100 nucleotides, and/or less than 5000, 2000, 1000, 500, 200, 100, 50, 20, or 10 nucleotides, among others. Alternatively, or in addition, the spacer may have a length that is greater than, or less than, the length of one or both targets. A longer spacer may be advantageous to size larger fragments more accurately, and a shorter spacer to size smaller fragments more accurately. In some examples, targets B and W may be attached directly to one another without any intervening spacer.

Each partition 254a-254c contains a pair of primers for amplification of each target (see FIGS. 4-6). Primers 274a, 274b are a forward primer and a reverse primer, respectively, to prime amplification of target B for formation of a first amplicon 256. Primers 276a, 276b are a forward primer and a reverse primer, respectively, to prime amplification of target W for formation of a second amplicon 258. The occurrence of each amplification reaction using one pair of primers is indicated by an amplifying arrow 252.

Each partition 254a-254c also contains a pair of optical probes to enable detection of the occurrence of each amplification reaction. A first probe 278 is configured to hybridize to target B and/or first amplicon 256, and a second probe 280 is configured to hybridize to target W and/or second amplicon 258. Each probe includes a fluorophore 282a or 282b and a quencher 284a or 284b for the fluorophore. Generation of first amplicon 256 separates fluorophore 282a from quencher 284a to increase a first fluorescence 262 of fluorophore 282a. Generation of second amplicon 258 separates fluorophore 282b from quencher 284b to increase a second fluorescence 264 of fluorophore 282b. In the depicted example, the separation of fluorophore and quencher from one another occurs by cleavage of first probe 278 or second probe 280, to generate a degraded probe 278d or 280d. In other examples, the separation may occur without degradation when the probe hybridizes to the corresponding amplicon (e.g., if the probe is a molecular beacon probe or a strand-displacement probe). In some examples, fluorophore 282a and fluorophore 282b may be identical to one another and distinguishable by their fluorescence intensities. In other examples, the probes may be replaced by an intercalating dye.

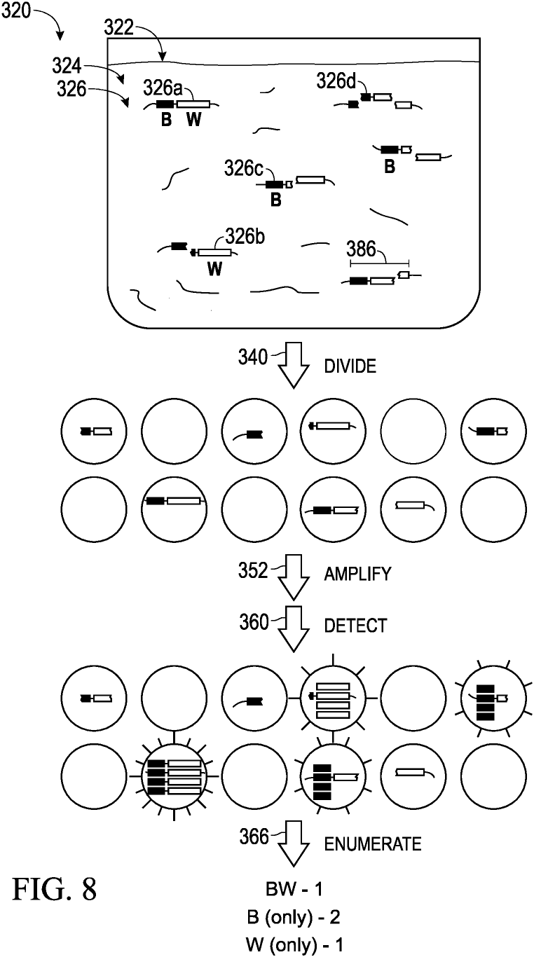

Partitions 254a-254c have distinguishable fluorescence patterns. Partition 254a contains both targets B and W and thus exhibits fluorescence of fluorophores 282a and 282b (see FIG. 4). Partition 254b contains target W but only a broken part of target B and thus exhibits fluorescence of fluorophore 282b but not fluorophore 282a (see FIG. 5). Partition 254c contains target B but only a broken part of target W and thus exhibits fluorescence of fluorophore 282a but not fluorophore 282b (see FIG. 6).

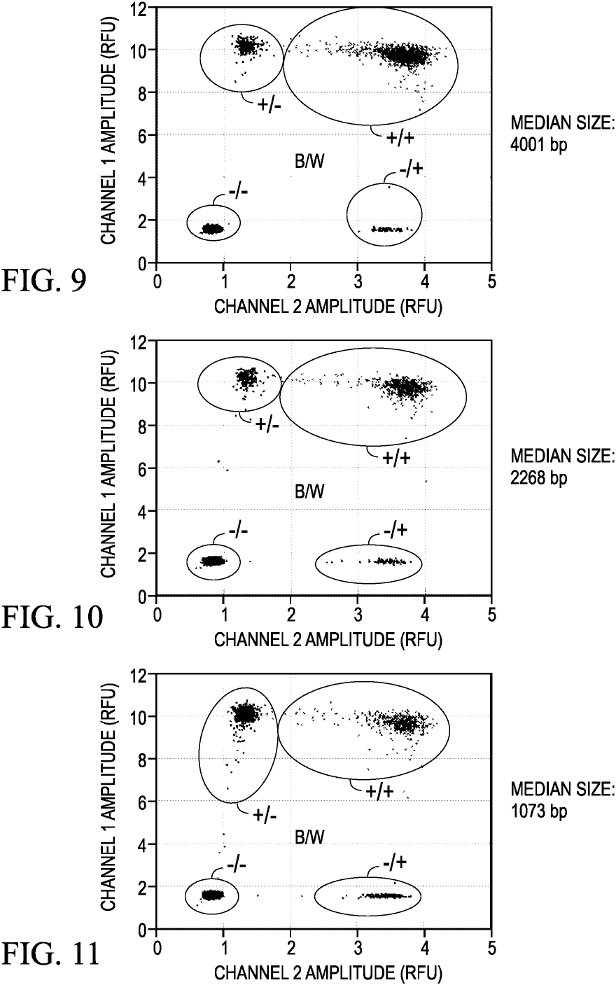

FIG. 7 shows a schematic flow diagram 220 for an assay on a sample-containing fluid 222 to predict at least one length characteristic of a population 224 of nucleic acid fragments 226 therein (compare with FIG. 1). Nucleic acid fragments 226 include a population of target-containing fragments 226a that provides copies of first target 228 (B) and second target 230 (W) (also see FIG. 4). Only a subset of nucleic acid fragments 226 contains targets B and W. In most cases, a large majority of population 224 is composed of target-negative fragments 232, but only a small number of target-negative fragments 232 are depicted in FIG. 7 for simplification. The mean/median length of nucleic acid fragments 226 is much larger than the length of a target region 286 of target-containing fragments 226a, where target region 286 is defined by targets B and W and spacer 272 (if present). Accordingly, none of the copies of target region 286 is broken. After dividing 240, amplifying 252, and detecting 260, enumerating 266 obtains partitions counts of B+W positive partitions, B-positive only partitions, and W-positive only partitions of 4, 0, and 0, respectively. A low percentage of B-positive only partitions and W-positive only partitions indicates that the median/mean length of nucleic acid fragments 226 in population 224 is large relative to the length of target region 286.

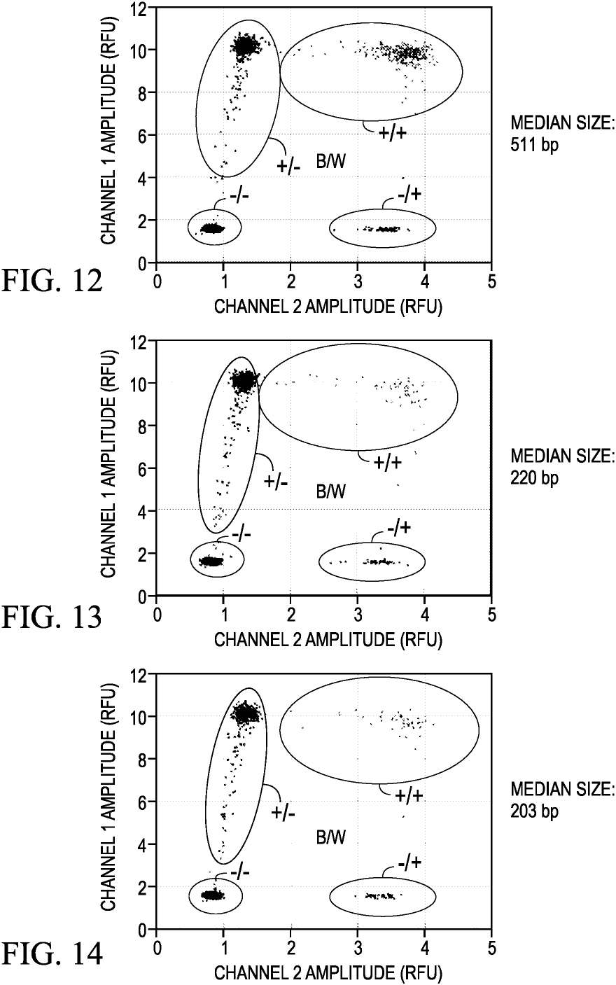

FIG. 8 shows a schematic flow diagram 320 for an assay on a sample-containing fluid 322 to predict at least one length characteristic of a population 324 of nucleic acid fragments 326 therein (compare with FIGS. 1, 2, and 7). Population 324 has a much smaller mean/median length than population 224 of FIG. 7. As a result, a target region 386 (B+W) provided by nucleic acid fragments 326 is frequently broken. (Target region 386 is identical in sequence to target region 286 of FIGS. 4 and 7.) In this highly simplified example, only target-containing fragment 326a has unbroken targets B and W. Target-containing fragment 326b has an unbroken target W but only a broken part of target B. Target-containing fragment 326c has an unbroken target B but only a broken part of target W. Partial target-containing fragment 326d has only part of each target B and W. After dividing 340, amplifying 352, and detecting 360, enumerating 366 obtains partition counts of B+W positive partitions, B-positive only partitions, and W-positive only partitions of 1, 2, and 1, respectively. The higher percentage of B-positive only partitions and W-positive only partitions results because population 324 is substantially more fragmented than population 224 of FIG. 7, such that the median/mean length of nucleic acid fragments 326 in population 324 is comparable to or less than the length of target region 386.

The partition counts for the three target-defined subsets of partitions (B+W, B-only, and W-only) may directly or indirectly provide inputs for respective independent variables of a size prediction algorithm. A linear combination of these partition counts, or values derived therefrom, by the size prediction algorithm can produce an output(s) representing at least one length characteristic of a population of nucleic acid fragments in a sample. Accordingly, in some examples, exactly three values (three partition counts or three values derived respectively therefrom) may be used as inputs sufficient for the size prediction algorithm to produce the output.

### C. Exemplary Amplification Data for Generating a Size Prediction Algorithm

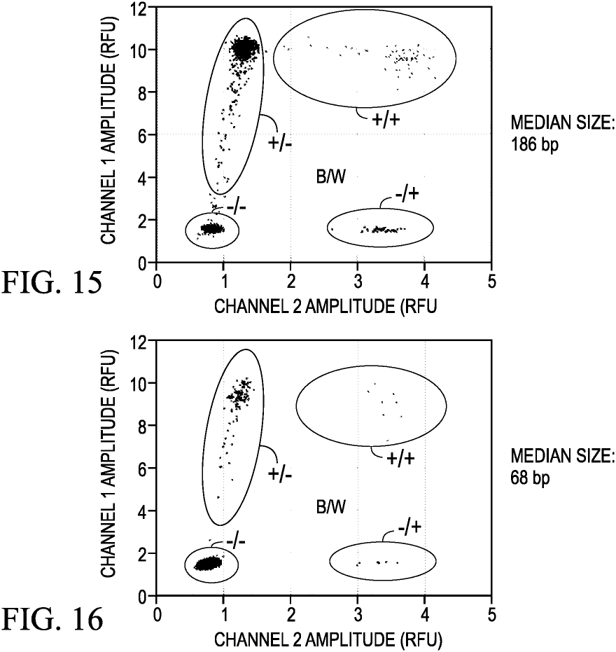

This subsection describes exemplary amplification data collected from a series of standards using the target and reagent configuration of Subsection B in the digital assay of Subsections A and B; see FIGS. 9-17. The amplification data demonstrate changes in the relative abundance of linked (B+W) and unlinked targets (B-only or W-only) as a function of median fragment length, and may be used for generating a size prediction algorithm, such as by machine learning.

FIGS. 9-16 are scatter plots of amplification data collected from a digital amplification assay performed in droplets (as the partitions), using the target and reagent configuration of FIGS. 4-6. Target B is 79 bp, target W is 200 bp, and the spacer separating the B and W targets is 9 bp. The digital amplification assay was conducted on each standard of a series of standards generated by fragmenting aliquots of a sample of genomic HEK cell DNA using Benzonase® Endonuclease purchased from MilliporeSigma, Burlington, MA. The median length of nucleic acid fragments in each standard is indicated for each plot and was measured independently by electrophoresis using a Bioanalyzer Instrument commercially available from Agilent, Santa Clara, CA.

The scatter plots represent each droplet as a point positioned according to the fluorescence amplitude (in relative fluorescence units (RFU)) measured from the droplet at each of two different wavelengths (“channel 1” and “channel 2”, respectively). The channel 1 fluorescence indicates whether the droplet contains at least one copy of target B, and the channel 2 fluorescence indicates whether the droplet contains at least one copy of target W. In each plot, four clusters of points/droplets are circled and identified according to B/W content: -/- (negative for B and W), +/- (positive for B only), -/+ (positive for W only), and +/+ (positive for both B and W).

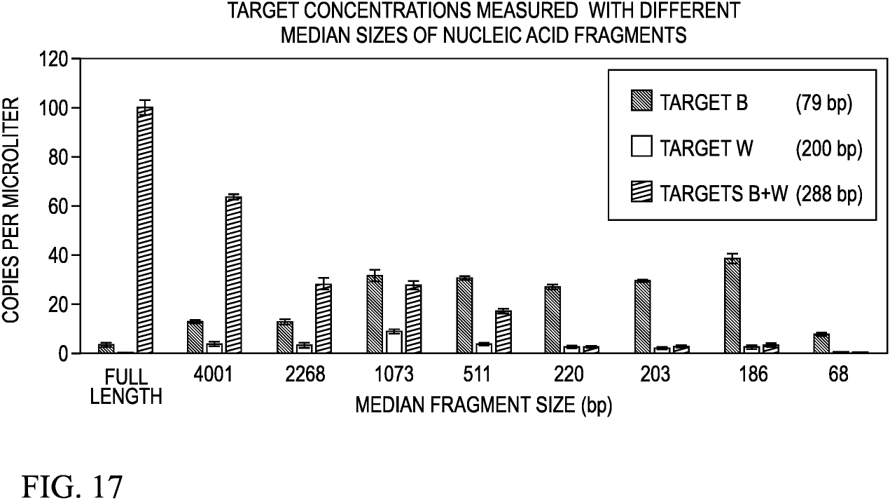

FIG. 17 shows a bar graph plotting the target concentrations (B-only fragments, W-only fragments, and B+W fragments) estimated from the scatter plots of FIGS. 9-16 for each standard of different median fragment length. With increasing fragmentation, the B+W concentration progressively decreases, the W-only concentration increases and then drops off, and the B-only concentration increases and then drops off more slowly than the W-only concentration.

A dataset composed of data points resulting from the assays of FIGS. 9-17, and the separately-measured length characteristic of each standard, may be utilized to estimate values for coefficients and a constant term of an input expression of a predictor function utilized by a size prediction algorithm. Each data point represents one of the standards. The digital amplification assay and a separate size measurement performed on the standard provide input values and an output value, respectively, for the data point. Any suitable enumeration values obtained in the amplification assays of FIGS. 9-16, or values derived therefrom, may be used as input values for data points of the dataset. The values for the coefficients and the constant term of the predictor function may be estimated by linear regression, logistic regression, or any other suitable statistical approach. The independently-measured median length (and/or other length characteristic) of each standard is the output value of the predictor function for each set of input values from one of the standards. The input values may, for example, be raw or normalized partition counts of target-defined subsets of partitions, estimated concentrations of each unlinked target and the linked targets, target ratios, fraction positive (or fraction negative) for a given target (unlinked) or a given combination of two or more linked targets, or the like.

### D. Evaluation of a Size Prediction Algorithm Generated by Logistic Regression

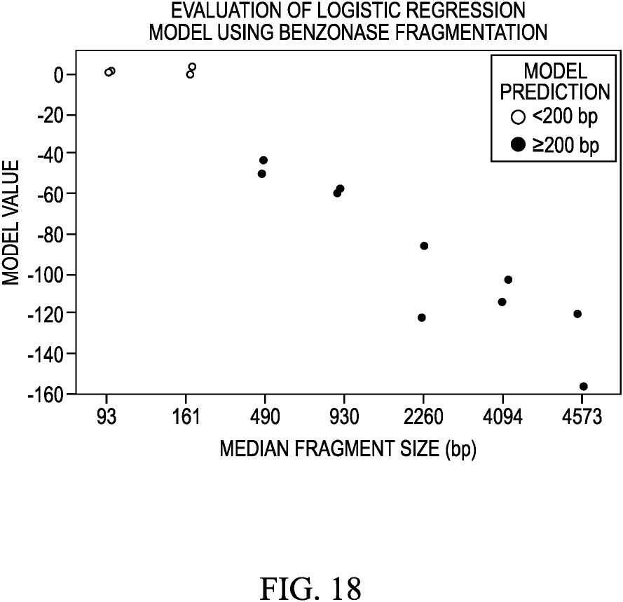

This subsection describes evaluation of a logistic regression model using a series of fragmented standards each having an independently-measured median length of nucleic acid fragments; see FIG. 18.

A series of standards were generated by treatment of genomic DNA with Benzonase® Endonuclease purchased from MilliporeSigma, Burlington, MA. The median length of nucleic acid fragments present in each standard was measured by electrophoresis using a Bioanalyzer Instrument commercially available from Agilent, Santa Clara, CA. Each standard has a different median fragment length. Each standard also was tested for the presence of unlinked target B, unlinked target W, and linked targets B+W in a digital amplification assay performed using droplets (as partitions), as described above in Subsections B and C. Raw counts of subsets of droplets positive for target B only, target W only, and targets B+W for each standard were used as inputs for a size prediction algorithm, which was generated in a logistic regression model by machine learning, as described generally in Subsection C. The size prediction algorithm provided an output (i.e., a model value) for each set of inputs (raw counts) from each standard. Each standard was tested in duplicate in the digital amplification assay, to obtain a pair of model (output) values for the standard.

FIG. 18 plots the pair of model (output) values for each standard according to the median fragment length in base pairs measured independently for the standard. The size prediction algorithm was generated by machine learning based on a 200 bp threshold median length. In other words, the coefficients and intercept of the size prediction algorithm were estimated such that an outputted model value below zero predicts a median fragment length greater than or equal to 200 bp, and a model value above zero predicts a median fragment length of less than 200 bp. Generally, the more negative the model value for a standard, the more certain that the median fragment length of the standard is greater than or equal to the threshold length of 200 bp. In the graph of FIG. 18, circles representing a model value above zero are unfilled, and circles representing a model value below zero are filled. Accordingly, the logistic regression model accurately predicts that the two most fragmented standards (i.e., 93 bp and 161 bp median fragment lengths) have a median fragment length of less than 200 bp. The logistic regression model also accurately predicts that the five least-fragmented standards (i.e., 490 bp, 930 bp, 2260 bp, 4094 bp, and 4573 bp median fragment lengths) have a median fragment length greater than or equal to 200 bp, with increasing certainty as the median fragment length increases. In some cases, samples that are degraded extensively can be more difficult to accurately size, because the degradation reduces the total number of detectable targets, which increases statistical variation. In these cases, shorter targets may be advantageous.

### E. Sizing Methods

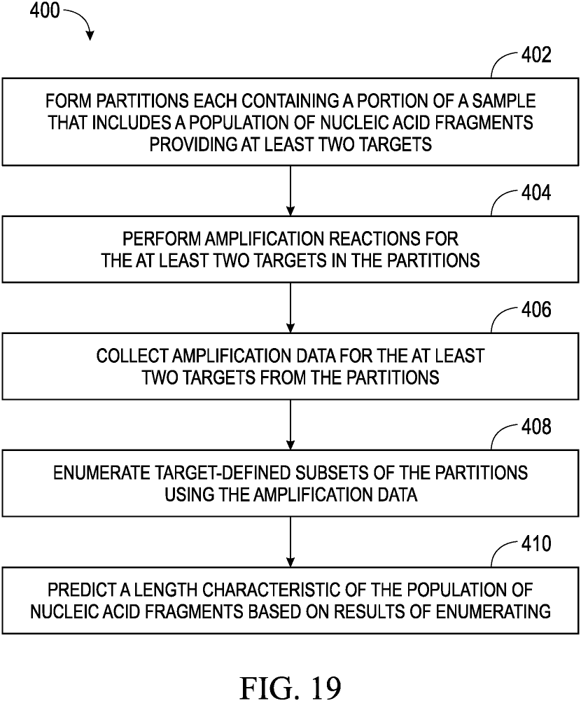

This subsection describes exemplary methods of sizing a population of nucleic acid fragments provided by a sample; see flowchart 400 of FIG. 19. Each method may include any suitable combination of the steps listed in flowchart 400, performed in any suitable order using any suitable aspects and features of the present disclosure (e.g., see Subsections A-D).

A set of partitions may be formed at step 402. Each partition may contain a portion of the sample, which includes a population of nucleic acid fragments. The nucleic acid fragments provide at least two targets, such as a first target and a second target, of different length from one another. The nucleic acid fragments may be nucleic acid polymers, such as DNA (e.g., genomic DNA), reverse-transcribed RNA (i.e., DNA resulting therefrom), RNA, or the like. The set of partitions may be formed by dividing a mixture that includes the sample and reagents sufficient for amplification of each target of the at least two targets. Alternatively, the sample may be divided separately from at least one of the amplification reagents and then combined with the at least one amplification reagent. In some cases, the nucleic acid fragments may provide at least one other target besides the first and second targets, such as a third target, a fourth target, and so on. Accordingly, the reagents for amplification also may be sufficient for amplification of the at least one other target.

Each target of the at least two targets may be present at partial occupancy in the set of partitions. In other words, only a subset of the partitions contains each target.

A pair (or more) of the targets may be linked at least partially to one another in the sample. This linkage causes the pair of targets to colocalize to the same partitions at a statistically significant frequency (i.e., greater than colocalization by chance). Breakage events that disrupt copies of a first target of the pair, copies of a second target of the pair, or copies of a spacer between the pair of targets, reduce the proportion of the first and second targets that are colocalized with one another in the partitions (see Subsections B and C).

Amplification reactions for the at least two targets may be performed in the set of partitions at step 404. The amplification reactions produce a respective amplicon corresponding to each target. A first amplicon may be produced only in partitions containing at least one copy of the first target, a second amplicon may be produced only in partitions containing at least one copy of the second target, and so on. The amplification reactions may be driven by heating and/or thermally cycling the set of partitions. Thermal cycling may drive a polymerase chain reaction or a ligase chain reaction, among others.

Amplification data for the at least two targets may be collected from the partitions at step 406. The amplification data results from the amplification reactions performed in step 404, and may be detected from the partitions serially or in parallel by any suitable technique. In some examples, the amplification data may be detected optically as fluorescence. Production of each amplicon may be optically distinguishable from production of each other amplicon in individual partitions by wavelength or intensity. In some cases, production of a first amplicon may be detected from a first label present in the set of partitions and production of a second amplicon may be detected from a second label. In some cases, production of each of the first and second amplicons may be detected from the same label (e.g., as a difference in intensity).

Target-defined subsets of the set of partitions may be enumerated at step 408 using the amplification data to obtain partitions counts. Enumeration may include determining a first partition count of a first subset of partitions positive for a first target, a second partition count of a second subset of partitions positive for a second target, a third partition count of a third subset of partitions positive for both the first target and the second target, a fourth partition count of a fourth subset of partitions negative for both the first target and the second target, and/or a total, target-independent count of the entire set of partitions. Determination of the total number of partitions allows the fraction of positive partitions or negative partitions for each target or target combination to be calculated, and a concentration of the target or target combination to be estimated from the fraction. The first subset of partitions may include all partitions positive for the first target or may be restricted to partitions positive for the first target and negative for the second target. The second subset of partitions may include all partitions positive for the second target or may be restricted to partitions positive for the second target and negative for the first target. Since positive and negative counts are inverse to one another, enumeration may include determining a count of partitions negative for the first target, a count of partitions negative for the second target, and, in some cases, a count of partitions negative for either of the first and second targets.

At least one length characteristic of the population of nucleic acid fragments may be predicted at step 410 based on results of enumerating (i.e., results from step 408). Each length characteristic may be predicted directly using partitions counts for each target and target combination or may be predicted using values derived from the partition counts. Exemplary derived values that may be suitable include fraction-positive/fraction-negative values for each target and/or target combination. Other exemplary derived values include normalized values, such as partition counts scaled by a scaling factor. Still other exemplary derived values that may be suitable include a concentration-related value (e.g., mean copies or moles per partition or unit volume) for each target and/or target combination. The concentration values may be calculated using a fraction-positive or fraction-negative value for each target and/or target combination. Yet other exemplary derived values that may be suitable include one or more target-positive ratios and/or target-negative ratios of targets/target combinations to one another.

Each length characteristic may be predicted using a size prediction algorithm. The size prediction algorithm may be generated by machine learning as described elsewhere herein. Input values for the size prediction algorithm may, for example, be any of the partition counts, normalized values, fraction-positive/fraction-negative values, concentration values, or target ratio values described above.

### F. Methods of Generating a Size Prediction Algorithm

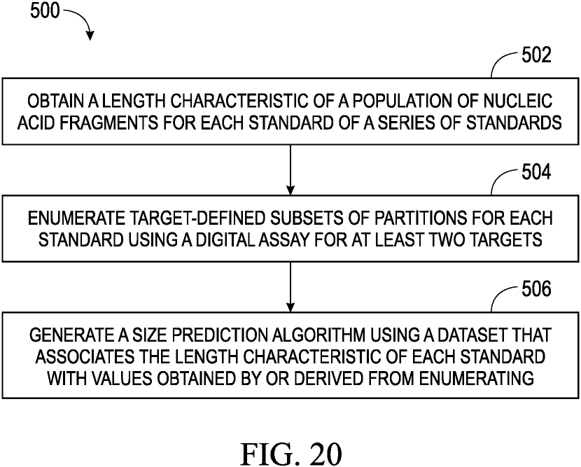

This subsection describes exemplary methods of generating a size prediction algorithm; see flowchart 500 of FIG. 20. Each method may include any suitable combination of the steps listed in flowchart 500, performed in any suitable order using any suitable aspects and features of the present disclosure.

At least one length characteristic of a population of nucleic acid fragments may be obtained at step 502 for each standard of a series of standards. The at least one length characteristic may be obtained by any suitable measurement technique, such as electrophoresis, mass spectrometry, flow cytometry, electron microscopy, or the like. Each standard may represent a different degree of nucleic acid fragmentation. Each standard may be generated by physical or chemical breakage of nucleic acid polymers. In some examples, more than one length characteristic may be obtained for each standard. For example, a median length and a percentage of fragments above/below a threshold size may be obtained for each standard.

The series of standards of step 502 also may be tested using a digital assay. A respective set of partitions for each standard may be formed, with each partition of the set including a portion of the standard. At least two targets (i.e., the same at least two targets) may be amplified in each set of partitions, and amplification data for the at least two targets may be collected from each set of partitions.

Target-defined subsets of partitions of each set of partitions may be enumerated at step 504. Enumeration may be conducted as described above in Subsection E for step 408 and may obtain any suitable partition count values and/or values derived therefrom.

A size prediction algorithm may be generated at step 506 using at least one dataset including data points resulting from steps 502 and 504 and corresponding to the series of standards. Each data point is defined by one or more input values for independent variables and an output value for a dependent variable. The input values are obtained by or derived from enumerating (i.e., from step 504) for one of the standards. The output value is obtained by or derived from measuring at least one length characteristic (i.e., from step 502) of the same standard. Generating the size prediction algorithm may be performed by linear regression or logistic regression (e.g., binary regression), among others. Generating the size prediction algorithm may include estimating values for coefficients of variable terms and a constant term of a regression model by machine learning. The values for the coefficients may include a weighting value for each of one, two, three, or more independent variables (model inputs) and/or an intercept value for a y-intercept. Any of the values or derived values described above in Subsection E in connection with step 408 may be suitable for generating the size prediction algorithm.

In some cases, at least two datasets may be used to generate the size prediction algorithm or at least two size prediction algorithms to predict at least two different length characteristics of a sample. The at least two datasets may result from the same series of standard or from different series of standards.

### IV. Illustrative Combinations and Additional Examples

This section describes additional aspects and features of the methods, systems, and computer-readable media of the present disclosure, presented without limitation as a series of paragraphs, some or all of which may be alphanumerically indexed for clarity and efficiency. Each of these paragraphs can be combined with one or more other paragraphs, and/or with disclosure from elsewhere in this application, in any suitable manner. Some of the paragraphs below expressly refer to and further limit other paragraphs, providing without limitation examples of some of the suitable combinations.

A1. A method of sizing a population of nucleic acid fragments provided by a sample, the method comprising: forming partitions each containing a portion of the sample, wherein each target of at least two targets is present in only a fraction of the nucleic acid fragments, and wherein, optionally, the at least two targets have different lengths relative to one another; performing amplification reactions for each of the at least two targets in the partitions; collecting amplification data for the at least two targets from the partitions; enumerating target-defined subsets of the partitions using the amplification data; and predicting at least one length characteristic of the population of nucleic acid fragments using results of enumerating.

A2. The method of paragraph A1, wherein predicting includes predicting whether the population of nucleic acid fragments meets one or more predefined size criteria.

A3. The method of paragraph A2, wherein predicting includes classifying a length of the population with respect to a threshold size.

A4. The method of paragraph A3, wherein predicting includes predicting whether the population of nucleic acid fragments has a median length or a mean length above or below the threshold size.

A5. The method of paragraph A4, wherein predicting includes computing a log odds that the population of nucleic acid fragments has the median length or the mean length above or below the threshold size.

A6. The method of any of paragraphs A1 to A5, wherein predicting includes predicting a value for a percentage of the population of nucleic acid fragments that meets one or more predefined size criteria.

A6.1. The method of paragraph A6, wherein the value for the percentage represents a percentage of the population of nucleic acid fragments that has a predefined relationship to a threshold length.

A6.2. The method of paragraph A6.1, wherein the threshold length is 200 base pairs or nucleotides.

A6.3. The method of claim A6.1 or A6.2, wherein the at least two targets include a first target and a second target, and wherein the first target is longer than the second target and has a length that is within 10%, 5%, or 2% of (or equal to) the threshold length.

A6.4. The method of any of paragraphs A6 to A6.3, wherein predicting includes predicting a confidence interval of the value for the percentage.

A7. The method of any of paragraphs A1 to A6, wherein predicting includes predicting a mean, median, quartile, and/or quintile length of the population of nucleic acid fragments.

A7.1. The method of any of paragraphs A1 to A7, wherein predicting include predicting a median length of nucleic fragments in the population.

A7.2. The method of paragraph A7.1, wherein predicting includes predicting a confidence interval of the median length.

A7.3. The method of paragraph 7.2, wherein the confidence interval is a 95% confidence interval of the median length.

A8. The method of any of paragraphs A1 to A7, wherein enumerating includes obtaining two or more enumeration values, and wherein predicting includes combining the two or more enumeration values, or values derived therefrom, as input values for independent variables of a predictor function, to generate an output value representing a length characteristic of the at least one length characteristic.

A9. The method of paragraph A8, wherein combining includes linearly combining the enumeration values, or respective values derived therefrom, with one another according to the predictor function, and wherein linearly combining includes multiplying each of the enumeration values, or each of the values derived therefrom, by a respective coefficient and summing products of multiplying.

A10. The method of paragraph A9, wherein the respective coefficients are not equal to one another.

A11. The method of paragraph A9 or A10, wherein none of the respective coefficients is equal to unity.

A12. The method of any of paragraphs A9 to A11, wherein summing the products of multiplying includes summing the products of multiplying and a constant with one another, and wherein the constant is an intercept of the predictor function.

A13. The method of any of paragraphs A8 to A12, wherein predicting includes combining at least three enumeration values, or respective values derived therefrom, with one another according to the predictor function.

A14. The method of paragraph A13, wherein predicting includes linearly combining exactly three enumeration values, or exactly three respective values derived therefrom, according to the predictor function.

A15. The method of paragraph A14, wherein the at least three enumeration values are obtained, directly or indirectly, from target-defined subsets of the partitions that are defined with respect to positivity/negativity of only a first target and a second target of the at least two targets.

A16. The method of any of paragraphs A1 to A15, wherein predicting is performed by a size prediction algorithm that utilizes a linear predictor function.

A17. The method of any of paragraphs A1 to A16, wherein enumerating includes obtaining a first partition count from a first subset of the partitions, or from an inverse of the first subset of the partitions, and a second partition count from a second subset of the partitions, or from an inverse of the second subset of the partitions, wherein the first subset of the partitions is positive for a first target of the at least two targets, wherein the second subset of the partitions is positive for a second target of the at least two targets, and wherein predicting uses the first partition count and the second partition count, or one or more values derived therefrom, as input values for independent variables of a predictor function to produce an output value representing a length characteristic of the at least one length characteristic.

A18. The method of paragraph A17, wherein the first subset of the partitions is negative for the second target, and wherein the second subset of the partitions is negative for the first target.

A19. The method of paragraph A17 or A18, wherein predicting uses the first partition count and the second partition count as direct inputs for the predictor function.

A20. The method of any of paragraphs A17 to A19, wherein predicting uses values derived respectively from the first and second partition counts as inputs for the predictor function.

A21. The method of paragraph A20, wherein predicting uses one or more target concentration values, fraction-positive/fraction-negative values, normalized values, and/or target ratio values derived from the first and second partition counts, as inputs for the predictor function.

A22. The method of paragraph A21, wherein predicting uses fraction-positive values or fraction-negative values each calculated with one of the first and second partition counts and a total, target-independent count of the partitions.

A23. The method of paragraph A21, wherein predicting uses a target ratio value calculated with the first and second partition counts.

A24. The method of any of paragraphs A17 to A23, wherein the first target and the second target have a degree of linkage to one another in the sample, wherein the first subset of the partitions is negative for the second target, wherein the second subset of the partitions is negative for the first target, wherein enumerating includes obtaining a third partition count from a third subset of the partitions that is positive for the first target and the second target, or from an inverse of the third subset of the partitions, and wherein predicting uses the first partition count, the second partition count, and the third partition count, or values derived therefrom, as inputs for independent variables of the predictor function.

A25. The method of paragraph A24, wherein predicting uses three inputs for the predictor function.

A26. The method of paragraph A24 or A25, wherein predicting uses the first, second, and third partition counts as direct inputs for the predictor function.

A27. The method of paragraph A24 or A25, wherein predicting uses one or more values derived from the first, second, and third partition counts as inputs for the predictor function.

A28. The method of any of paragraphs A24 to A27, wherein the sample represents a genome in which the first target and the second target are separated from one another by less than 200 base pairs.

A29. The method of any of paragraphs A24 to A28, wherein the degree of linkage of the first target and the second target to one another in the sample is related to a median length of the population of nucleic acid fragments.

A30. The method of any of paragraphs A1 to A29, wherein predicting utilizes a predictor function having coefficient values estimated from a dataset including a plurality of data points, wherein each data point represents a standard of a series of standards having different amounts of nucleic acid fragmentation, wherein the data point includes input values for two or more independent variables of the predictor function and an output value for a dependent variable of the predictor function, wherein the values for the two or more independent variables of the predictor function are derived from amplification data for the at least two targets collected in a digital assay from a set of partitions each containing a portion of the standard, and wherein the output value for the dependent variable of the predictor function results from measuring a length of nucleic fragments of the standard separately from the digital assay.

A31. The method of paragraph A30, wherein the coefficient values were estimated using logistic regression.

A32. The method of paragraph A30, wherein the coefficient values were estimated using linear regression.

A33. The method of any of paragraphs A1 to A32, wherein the at least two targets include a first target and a second target, and wherein the second target is at least 25%, 50%, 75%, or 100% longer than the first target.

A34. The method of any of paragraphs A1 to A33, wherein the at least two targets include a first target and a second target, wherein forming includes forming the partitions to each contain a first probe for detecting amplification of the first target and a second probe for detecting amplification of the second target, and wherein the first probe includes a first label and the second probe includes a second label.

A35. The method of paragraph A34, wherein the first label and the second label include different fluorophores from one another.

A36. The method of paragraph A35, wherein collecting amplification data includes detecting fluorescence from the first label and the second label, wherein enumerating with respect to the first target is based on fluorescence from the first label, and wherein enumerating with respect to the second target is based on fluorescence from the second label.

A37. The method of any of paragraphs A1 to A36, wherein the at least two targets include a first target and a second target, and wherein forming includes forming the partitions to each contain a first pair of primers for amplifying the first target and a second pair of primers for amplifying the second target.

A38. The method of any of paragraphs A1 to A37, wherein the at least two targets include a first target and a second target, and wherein the second target is at least 25, 50, 75, or 100 nucleotides longer than the first target.

A39. The method of any of paragraphs A1 to A38, wherein the at least two targets include a first target and a second target, wherein the sample represents a genome, and wherein the first target and the second target are separated from one another by less than 10, 5, 2, 1, 0.5, 0.2, 0.1, 0.05, or 0.02 kilobase(s) in the genome.

A40. The method of any of paragraphs A1 to A39, wherein the nucleic acid fragments include fragments of genomic DNA, RNA, or reverse-transcribed RNA.

A41. The method of any of paragraphs A1 to A40, wherein the at least two targets include a first target and a second target, and wherein the first target is longer than the second target and has a length that is within 10%, 5%, or 2% of 200 nucleotides.

B1. A method of obtaining a size prediction algorithm for sizing a population of nucleic acid fragments in a sample, the method comprising: providing a series of standards, each standard of the series having a different degree of nucleic acid fragmentation and including at least two targets; forming sets of partitions, each set of partitions containing portions of one of the standards and containing each of the at least two targets at partial occupancy; performing amplification reactions for each of the least two targets in each set of partitions; collecting amplification data for the at least two targets from each set of partitions; enumerating target-defined subsets of each set of partitions using the amplification data; measuring, for each standard, a length of nucleic acid fragments present in the standard; and generating the size prediction algorithm based on results of enumerating and measuring.

B2. The method of paragraph B1, wherein generating includes estimating values for coefficients and an intercept of a predictor function utilized by the size prediction algorithm.

B3. The method of paragraph B2, wherein generating uses values obtained by, or derived from, enumerating as input values for independent variables of the predictor function, and wherein measuring uses values obtained by, or derived from, measuring as output values for an independent variable of the predictor function.

B4. The method of paragraph B2 or B3, wherein generating includes estimating the values for the coefficients and the intercept by logistic regression.

B5. The method of any of paragraphs B1 to B3, wherein generating includes estimating the values for the coefficients and the intercept by linear regression.

B6. The method of any of paragraphs B1 to B5, wherein measuring includes performing electrophoresis on nucleic acid fragments of each standard.

B7. The method of any of paragraphs B1 to B6, further comprising fragmenting nucleic acid polymers to generate each standard.

B8. The method of paragraph B7, wherein fragmenting includes chemically fragmenting the nucleic acid polymers.

B9. The method of paragraph B8, wherein chemically fragmenting includes performing an enzyme-catalyzed chemical reaction to break the nucleic acid polymers.

B10. The method of paragraph B7, wherein fragmenting includes physically breaking the nucleic acid polymers.

C1. A computer-readable medium containing program instructions for sizing nucleic acid fragments in a sample, wherein execution of the program instructions by one or more processors of a computer system causes the one or more processors to perform a method comprising: predicting at least one length characteristic of a population of nucleic acid fragments using values obtained by, or derived from, enumerating target-defined subsets of partitions in a digital amplification assay for at least two targets, the partitions containing each of the at least two targets at partial occupancy, each of the at least two targets being provided by the population of nucleic acid fragments.

C2. The computer-readable medium of paragraph C1, wherein the computer-readable medium is non-transitory.

C3. The computer-readable medium of paragraph C1, wherein the computer-readable medium is transitory.

C4. The computer-readable medium of any of paragraphs C1 to C3, wherein predicting uses a first partition count and a second partition count, or one or more values derived therefrom, as inputs for a size prediction algorithm, wherein the first partition count represents a first subset of the partitions, or represents an inverse of the first subset of the partitions, and the second partition count represents a second subset of the partitions, or represents an inverse of the second subset of the partitions, wherein the first subset of the partitions is positive for a first target of the at least two targets, and wherein the second subset of the partitions is positive for a second target of the at least two targets.

C5. The method of paragraph C4, wherein the first subset of the partitions is negative for the second target, and wherein the second subset of the partitions is negative for the first target.

C6. The method of paragraph C4 or C5, wherein predicting uses the first partition count and the second partition count as direct inputs for the size prediction algorithm.

C7. The method of paragraph C4 or C5, wherein predicting uses one or more values derived from the first and second partition counts as inputs for the size prediction algorithm.

C8. The method of paragraph C7, wherein predicting uses one or more target concentration values, normalized values, and/or fraction-positive/fraction-negative values derived from the first and second partition counts, as inputs for the size prediction algorithm.

C9. The method of paragraph C8, wherein predicting uses fraction-positive values each calculated with one of the first and second partition counts and a total, target-independent count of the partitions, as inputs for the size prediction algorithm.

C10. The method of any of paragraphs C4 to 9, wherein the first target and the second target have a degree of linkage to one another in the sample, wherein the first subset of the partitions is negative for the second target, wherein the second subset of the partitions is negative for the first target, wherein a third partition count represents a third subset of the partitions positive for the first target and the second target, or represents an inverse of the third subset, and wherein predicting uses the first partition count, the second partition count, and the third partition count, or values derived therefrom, as inputs for the size prediction algorithm.

C11. The method of any of paragraphs C1 to C10, wherein predicting uses three inputs, optionally exactly three inputs, for the size prediction algorithm.

C12. The method of paragraph C10 or C11, wherein predicting uses the first, second, and third partition counts as direct inputs for the size prediction algorithm.

C13. The method of paragraph C10 or C11, wherein predicting uses one or more values derived from the first, second, and third partition counts, as inputs for the size prediction algorithm.

C14. The method of any of paragraphs C1 to C13, wherein predicting includes predicting a mean, median, quartile, and/or quintile length of the population of nucleic acid fragments.

C15. The method of any of paragraphs C1 to C14, wherein predicting includes predicting whether the population of nucleic acid fragments has a median length or a mean length above or below a threshold size.

C16. The method of paragraph C15, wherein predicting includes computing a log odds that the population of nucleic acid fragments has the median length or the mean length above or below the threshold size.

C17. The computer-readable medium of any of paragraphs C1 or C16, wherein predicting is performed using a size prediction algorithm generated by logistic regression.

C18. The computer-readable medium of any of paragraphs C1 to C16, wherein predicting is performed using a size prediction algorithm generated by linear regression.

C19. The computer-readable medium of any of paragraphs C1 to C18, wherein predicting is performed using a size prediction algorithm generated using quantification data obtained for each target of the at least two targets from each standard of a series of standards representing different amounts of nucleic acid fragmentation.

C20. The computer-readable medium of paragraph C19, wherein the size prediction algorithm was generated using a dataset including the quantification data and a length characteristic of each standard of the series of standards.

D1. A system for sizing a population of nucleic acid fragments, the system comprising: a first primer pair for amplifying a first target; a second primer pair for amplifying a second target; and a computer-readable medium containing program instructions executable by one or more processors of a computer system to predict at least one length characteristic of the population of nucleic acid fragments using values obtained or derived from enumerating target-defined subsets of partitions in a digital amplification assay, each partition containing the first primer pair and the second primer pair, the partitions containing each of the first target and the second target at partial occupancy, the first target and the second target being provided by the population of nucleic acid fragments.

D2. The system of paragraph D1, further comprising a first probe to detect amplification of the first target using the first primer pair and a second probe to detect amplification of the second target using the second primer pair.

### V. Advantages and Benefits

The different examples of sizing methods and systems, and associated computer-readable media, provide several advantages over known solutions for sizing a population of nucleic acid fragments. For example, illustrative examples described herein allow prediction of a length characteristic of nucleic acid fragments in a sample that is more sensitive, uses less sample, and/or is more suitable for automation.

Additionally, and among other benefits, illustrative examples described herein leverage machine learning to generate a size prediction algorithm that is more accurate than other prediction strategies.

Additionally, and among other benefits, illustrative examples described herein predict a length characteristic of a sample from one or more inputs generated by a digital assay performed on the sample, without the need for electrophoresis of the sample.

Additionally, and among other benefits, illustrative examples described herein generate or use a size prediction algorithm that predicts a length characteristic from two or more inputs, and in some cases, from a greater number of inputs than labels to detect target amplification.

Additionally, and among other benefits, illustrative examples described herein predict a length characteristic of a sample based on digital amplification of two or more targets, which offers high sensitivity.

Additionally, and among other benefits, illustrative examples described herein predict both a length characteristic of a population of nucleic fragments of a sample and a target quantification for the sample based on digital amplification of two or more targets.

Additionally, and among other benefits, illustrative examples described herein detect the presence of two linked targets of different length, and colocalization of the linked targets to the same partitions provides an additional input for a size prediction algorithm.

Additionally, and among other benefits, illustrative examples described herein provide an assay for the extent of nucleic acid degradation (DNA and/or RNA) of a sample. Accordingly, the assay may permit a user to identify excessively degraded samples and exclude these samples from other testing.

No known system or method can perform these functions. However, not all examples described herein provide the same advantages or the same degree of advantage.

### VI. Conclusion

The disclosure set forth above may encompass multiple distinct examples with independent utility. Although each of these has been disclosed in its preferred form(s), the specific examples thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

We claim:

1. A method of sizing a population of nucleic acid fragments provided by a sample, the method comprising:
forming partitions each containing a portion of the sample, wherein each target of at least two targets is present in only a fraction of the nucleic acid fragments;
performing amplification reactions for each of the at least two targets in the partitions;
collecting amplification data for the at least two targets from the partitions; enumerating target-defined subsets of the partitions using the amplification data; and
predicting at least one length characteristic of the population of nucleic acid fragments using results of enumerating.

2. The method of claim 1, wherein predicting includes predicting whether the population of nucleic acid fragments meets one or more predefined size criteria.

3. The method of claim 2, wherein predicting includes classifying a length of the population with respect to a threshold size.

4. The method of claim 1, wherein predicting includes predicting a value for a percentage of the population of nucleic acid fragments that meets one or more predefined size criteria.

5. The method of claim 4, wherein the value for the percentage represents a percentage of the population of nucleic acid fragments having a predefined relationship to a threshold length.

6. The method of claim 5, wherein predicting includes predicting a confidence interval of the value for the percentage.

7. The method of claim 1, wherein predicting includes predicting a mean, median, quartile, and/or quintile length of the population of nucleic acid fragments.

8. The method of claim 7, wherein predicting includes predicting a median length of nucleic fragments in the population.

9. The method of claim 8, wherein predicting includes predicting a confidence interval of the median length.

10. The method of claim 1, wherein enumerating includes obtaining two or more enumeration values, and wherein predicting includes combining the two or more enumeration values, or values derived therefrom, as input values for independent variables of a predictor function, to generate an output value representing a length characteristic of the at least one length characteristic.

11. The method of claim 10, wherein combining includes linearly combining the enumeration values, or respective values derived therefrom, with one another according to the predictor function, and wherein linearly combining includes multiplying each of the enumeration values, or each of the values derived therefrom, by a respective coefficient and summing products of multiplying.

12. The method of claim 11, wherein predicting includes combining at least three enumeration values, or respective values derived therefrom, with one another according to the predictor function.

13. The method of claim 12, wherein the at least three enumeration values are obtained, directly or indirectly, from target-defined subsets of the partitions that are defined with respect to positivity/negativity of only a first target and a second target of the at least two targets.

14. The method of claim 1, wherein enumerating includes obtaining a first partition count from a first subset of the partitions, or from an inverse of the first subset of the partitions, and a second partition count from a second subset of the partitions, or from an inverse of the second subset of the partitions, wherein the first subset of the partitions is positive for a first target of the at least two targets, wherein the second subset of the partitions is positive for a second target of the at least two targets, and wherein predicting uses the first partition count and the second partition count, or one or more values derived therefrom, as input values for independent variables of a predictor function to produce an output value representing a length characteristic of the at least one length characteristic.

15. The method of claim 14, wherein the first subset of the partitions is negative for the second target, and wherein the second subset of the partitions is negative for the first target, and wherein predicting uses the first partition count and the second partition count as direct inputs for the predictor function.

16. The method of claim 14, wherein predicting uses one or more target concentration values, fraction-positive/fraction-negative values, normalized values, and/or target ratio values derived from the first and second partition counts, as inputs for the predictor function.

17. The method of claim 14, wherein the first target and the second target have a degree of linkage to one another in the sample, wherein the first subset of the partitions is negative for the second target, wherein the second subset of the partitions is negative for the first target, wherein enumerating includes obtaining a third partition count from a third subset of the partitions that is positive for the first target and the second target, or from an inverse of the third subset of the partitions, and wherein predicting uses the first partition count, the second partition count, and the third partition count, or values derived therefrom, as inputs for independent variables of the predictor function.

18. The method of claim 1, wherein predicting utilizes a predictor function having coefficient values estimated from a dataset including a plurality of data points, wherein each data point represents a standard of a series of standards having different amounts of nucleic acid fragmentation, wherein the data point includes input values for two or more independent variables of the predictor function and an output value for a dependent variable of the predictor function, wherein the values for the two or more independent variables of the predictor function are derived from amplification data for the at least two targets collected in a digital assay from a set of partitions each containing a portion of the standard, and wherein the output value for the dependent variable of the predictor function results from measuring a length of nucleic fragments of the standard separately from the digital assay.

19. The method of claim 1, wherein the at least two targets include a first target and a second target, and wherein the second target is at least 50 nucleotides longer than the first target.

20. The method of claim 1, wherein the at least two targets include a first target and a second target, wherein the sample represents a genome, and wherein the first target and the second target are separated from one another by less than 100 nucleotides in the genome.

21. A method of obtaining a size prediction algorithm for sizing a population of nucleic acid fragments in a sample, the method comprising:
providing a series of standards, each standard of the series having a different degree of nucleic acid fragmentation and including at least two targets;
forming sets of partitions, each set of partitions containing portions of one of the standards and containing each of the at least two targets at partial occupancy;
performing amplification reactions for each of the least two targets in each set of partitions;
collecting amplification data for the at least two targets from each set of partitions;
enumerating target-defined subsets of each set of partitions using the amplification data;
measuring, for each standard, a length of nucleic acid fragments present in the standard; and
generating the size prediction algorithm based on results of enumerating and measuring.

22. The method of claim 21, wherein generating includes estimating values for coefficients and an intercept of a predictor function utilized by the size prediction algorithm.

23. The method of claim 22, wherein generating uses values obtained by, or derived from, enumerating as input values for independent variables of the predictor function, and wherein measuring uses values obtained by, or derived from, measuring as output values for an independent variable of the predictor function.

24. A computer-readable medium containing program instructions for sizing nucleic acid fragments in a sample, wherein execution of the program instructions by one or more processors of a computer system causes the one or more processors to perform a method comprising:
predicting at least one length characteristic of a population of nucleic acid fragments using values obtained by, or derived from, enumerating target-defined subsets of partitions in a digital amplification assay for at least two targets, the partitions containing each of the at least two targets at partial occupancy, each of the at least two targets being provided by the population of nucleic acid fragments.
